(19) United States
(12) Patent Application Publication
(10) Pub. No.: US 2008/0305550 A1
(43) Pub. Date: Dec. 11, 2008

(54) Targets for Detection of Ischemia

(76) Inventors: Hollie Huff, Denver, CO (US); Shannon George, Spokane, WA (US); Ping Wu, Aurora, CO (US)

Correspondence Address: SWANSON & BRATSCHUN, L.L.C., 8210 SOUTHPARK TERRACE, LITTLETON, CO, 80120, US

(21) Appl. No.: 11/814,044

(22) PCT Filed: Jan. 23, 2006
(86) PCT No.: PCT/US06/02327

§ 371 Date: Mar. 11, 2008

Related U.S. Application Data
(60) Provisional application No. 60/645,991, filed on Jan. 21, 2005.

(51) Int. Cl. G01N 33/68; G01N 33/50; G01N 33/20; C07K 16/18

(52) U.S. Cl. 436/71; 436/86; 436/73; 530/387.1; 530/387.9

(57) ABSTRACT

The subject application comprises methods for determining the occurrence of an ischemic event in a subject by determining an ischemia score based on the amount of at least two ischemia modified albumin markers. The ischemia modified albumin markers include complexes of fatty acids bound to albumin, albumin molecules with open Cys34 sites, albumin molecules that are products of oxidation at Cys34, albumin molecules with altered conformation or altered divalent metal binding due to the conformational change or oxidation at Cys34, and albumin molecules that have been oxidized at the N-terminus. Also included in the invention are ligands to each of the foregoing ischemia modified albumin markers. Further included are methods of determining the occurrence of an ischemic event by determining the amount of fatty acid that is complexed to albumin in a patient sample. In another embodiment, an ischemic event is determined by quantitating the relative amounts of reduced and oxidized forms of albumin Cys34. In an additional embodiment, an ischemic event is determined by observing whether a shift in albumin conformation has occurred which would reflect oxidized Cys34. Further, the invention comprises a method of determining an ischemic event by determining the amount of metal ion bound to the albumin metal ion binding sites.

### FIELD OF INVENTION

This invention pertains to in vitro diagnostic devices using various detection methods for positive and negative indications of ischemia. In particular, this invention uses various modifications of albumin, purification techniques and added reagents to detect differences in albumin as an indication of ischemia in a diagnostic test. Quantifying the ratios between different species of albumin provides not only positive and negative ischemic information, but also provides an indication of disease severity.

### BACKGROUND OF INVENTION

Ischemia is a physiological condition resulting from an imbalance between oxygen supply and demand in tissue. This condition typically arises due to an obstruction. The body may undergo periods of transient ischemia in which the obstruction is cleared, but prolonged conditions of ischemia may result in cell death (necrosis). Untreated ischemia can result in an acute myocardial infarction (AMI), which can be diagnosed by testing for a variety of physiological markers of necrosis. These markers include proteins, such as troponin and myoglobin, that are released from cells following cell death. It would be clinically beneficial to diagnose myocardial ischemia, before cell death, to allow treatment of the disease prior to irreversible damage. Currently, myocardial ischemia is diagnosed by electrocardiogram (ECG), echocardiography or myocardial perfusion imaging, which have poor accuracy and are high in cost compared to other classical diagnostic assays. More efficient health care management requires the rapid identification of ischemic patients so that non-ischemic patients can be released early to decrease overall treatment costs. A reliable, relatively inexpensive diagnostic assay for myocardial ischemia, utilizing a blood sample, would meet such demands.

The prior art describes a number of markers that are actual or purported markers for ischemia. These markers include ischemia modified albumin (e.g., U.S. Pat. No. 5,227,307 and WO 00/20840), free fatty acids (e.g., U.S. Pat. No. 6,750,030, WO 03/025571 and WO 03/024306), cardiac markers such as troponin, CK-MB and myoglobin (e.g., U.S. Pat. No. 5,710,008). While ischemia always precedes necrosis, it is not true that ischemia necessarily is followed by necrosis. Thus, the reliable identification of actual ischemia markers in patients prior to cell necrosis provides the physician with more information, thereby enabling appropriate treatment. Ischemia modified albumin is an actual marker of ischemia. Necrotic markers, such as troponin, CK-MB and myoglobin, are released from necrotic cells, and are not useful in detecting the earlier stage of ischemia prior to necrosis.

Albumin is the predominant protein in blood and is often thought of as a scavenger or sacrifice protein. It binds many other proteins, amino acids, drugs, metal ions and toxins that are ingested or released by the body during normal and abnormal conditions. Albumin is a critical protein in maintaining physiological homeostasis and as such can be used as an overall diagnosis of physiological well-being.

Myocardial ischemia results from an occlusion that prevents adequate blood supply to cardiac tissue, thereby depriving that tissue of needed oxygen. Occlusions result from arterial plaque build up which may activate inflammation pathways in addition to increased free radical generation as a result of decreased oxygen. Albumin scavenges many of the acute phase reactants by binding up proteins, peptides and ions released as a result of activation of inflammation pathways. Albumin also undergoes free radical damage, thereby deactivating the reactive molecules and protecting other important proteins and tissue from damage.

Divalent metal binding to albumin is well described in the literature. There are many metal binding sites on albumin with varying affinities. For example, copper has six described sites with the primary binding site being the N-terminus of albumin with a Kₐ=1.5×10¹⁶ (Masuoka, et al. (1993) The Journal of Biological Chemistry. 268(29): 21533-21537; Bar-Or, D. et al. (2001) Eur. Journal of Biochemistry 268:42-47). Subsequent binding sites become progressively less tightly bound (Zgirski A. et al. (1990) Journal of Inorganic Biochemistry 39(2): 137-48).

Tight binding of metal to the albumin requires multiple coordination sites, which can include numerous amino acids within the protein as well as H₂O. In particular, amino acids containing free amine, imidazole rings, carboxyl groups and sulfhydryl groups are capable of providing ligands for metal binding. Additionally, other proteins in human fluid that contain these amino acids may provide the ligands necessary for tight coordination complexation or independently bind creating a weaker bond. In a blood sample, albumin is the major contributor of free sulfhydryl groups (80-90%) because of its high concentration relative to other proteins and small numbers of molecules containing free thiols.

Albumin contains 35 cysteine residues (—SH groups). During posttranslational modifications, these —SH groups are oxidized to disulfide bonds (—S—S—), which stabilize the structure of albumin. The single cysteine at position 34 remains in the reduced form and at physiological pH is in its anionic form (S⁻). This free sulfhydryl group is blocked by mixed disulfide formation about 30% of the time. Reducing agents will reverse disulfide formation to yield native albumin and free the blocking agent for further characterization. Cysteine 34 on albumin is known to contribute to the conformation of the protein. If unoccupied, Cys34 remains as a free anion. Once a sulfhydryl group binds to Cys34, the conformation of the protein is changed, thereby reducing the metal binding affinity at the N-terminus (Zhang Y. et al. (2002) Journal of Biological Inorganic Chemistry 7(3):327-37). There are a number of circulating sulfhydryl containing molecules such as cysteine, homocysteine and glutathione that are scavenged by albumin. About 30% of healthy albumin is carrying cysteine bound at the Cys34 site (Peters, T, Jr. All about Albumin. Academic Press, Inc. 1996). During disease states or times of strenuous activity, the concentrations of circulating molecules containing sulfhydryl groups increase (Fellah, H. et al. (2003) Clinical Chemistry Laboratory 41(5): 675-80; Kelly, P. J. et al. (2004) Stroke 35(1):12-15; and Morgenstern, I. et al. (2003) Dig. Dis. Sci. 48(10):2083-90). The increase in these groups mean that there are more ligands available to bind the Cys34, thereby causing a conformational change that reduces the amount of metal binding affinity at the N-terminus. Under normal (i.e., non-ischemic) conditions, the Cys34 site remains protected in a clef of albumin (Peters, T, Jr. All about Albumin. Academic Press, Inc. 1996), but as albumin scavenges additional ligands such as long chain fatty acids, this site may become more susceptible to reactions with small molecules including other thiol containing molecules. The concentrations of ligands that affect the availability of the Cys34 site for binding new ligands also change with disease state.

WO 04/032711 describes methods for diagnosing or monitoring ischemia by determining post-translationally modified albumin, including cysteinylated-albumin or phosphorylated albumin. If the quantity of cys-albumin or phos-albumin is outside normal range, this may be diagnostic of ischemia. WO 04/032711 also purportedly describes the use of panels of phos-albumin, cys-albumin and other post-translationally modified proteins (organ or tissue-specific proteins) such as troponin or CK-MB, to determine if general ischemia is present and, if so, the location of the ischemia. WO 04/032711 also describes polyclonal antibodies specific to human cys-albumin.

In contrast to the prior art, the subject invention provides an indictor of disease states such as ischemia by monitoring the cascade of binding events of various ligands to albumin and the subsequent conformational changes of albumin. A clinician can use combinations of these events to differentiate among disease progression stages.

### SUMMARY OF THE INVENTION

Albumin altered during and following ischemia is termed ischemia modified albumin (IMA), and can be quantitatively measured for use as a diagnostic test. IMA may be measured by several methods including but not limited to mass spectrometry, HPLC, immunoassay, electrochemical and colorimetric techniques. This invention encompasses an understanding of the mechanism as a biological pathway, which has measurable markers that can be used as an indicator of disease severity. These measurable markers can be combined as a panel and used together as indication of the extent of disease progression. A physician can use this information to improve the care of the patient, prescribe more appropriate medications and monitor the progress of the patient.

In one embodiment, the subject invention comprises a method having the following steps. In a sample material obtained from a subject, at least two ischemia modified albumin markers are determined, i.e., detected or measured. These ischemia modified albumin markers include two or more of the following: complexes of fatty acids bound to albumin; molecules with open Cys34 sites; albumin molecules that are products of oxidation at Cys34; albumin molecules with altered conformation, or altered or reduced divalent metal binding due to the conformational change or oxidation at Cys34; and albumin molecules that have been oxidized at the N-terminus. Each of these markers can be determined in absolute terms or relative terms, i.e., as compared to albumin that has not been exposed to an ischemic event. Each of these markers is discussed in detail herein.

Next, the results of the at least two ischemia modified albumin markers described above are used to determine at least one ischemia score. The ischemia score can be determined using the ischemia modified albumin marker results alone or in combination with other clinical assays or observations. The ischemia score can be obtained by combining the ischemia marker results in an algorithm. Additionally, in calculating the ischemia score, each ischemia modified albumin marker result can be assigned an intensity factor which reflects or corresponds to the concentration of the marker. Further, each ischemia marker can be assigned a severity factor which is a value that reflects the weight that a particular marker has as an indicator of an ischemic event, as is further discussed herein.

Then, the ischemia score, or multiple ischemia scores obtained over time, can be used to determine the presence of an ischemic event in the subject. Alternatively, the ischemic event can be determined by the ischemia score(s) alone, or in combination with other clinical assays or observations. Determination of the ischemic event can be achieved by comparing the ischemia score(s) of the subject to a normal range, i.e., a range containing ischemic scores of a non-ischemic control population, and/or by comparison to ischemic score(s) of known ischemic patients.

The invention further comprises a method having the following steps. An entity (a person, computer software, diagnostic apparatus or the like) receives at least one ischemic score, which has been determined based at least in part on results of a determination, in a sample obtained from a subject, of at least two ischemia modified albumin markers. These markers can include at least two of the following: fatty acids bound to albumin; albumin molecules with open Cys34 sites; albumin molecules that are products of oxidation at Cys34; albumin molecules with altered conformation or altered or reduced divalent metal binding due to conformational change or oxidation at Cys34; and albumin molecules that have been oxidized at the N-terminus.

Ligands encompassed within the scope of the invention include antibodies (monoclonal, polyclonal or fragments thereof) that bind and are specific to: complexes of albumin and one or more fatty acids; an albumin molecule with an exposed Cys34 site; an albumin molecule that is a product of an oxidation reaction at Cys34; an albumin molecule that has an altered conformation or altered or reduced divalent metal binding due to the conformational change at Cys34; and an albumin molecule that has 2-oxo-histidine at His3 and/or His9. Monoclonal and polyclonal antibodies are obtained using methods well-known to the skilled artisan. When the antibodies are specific to albumin that has been oxidized at Cys34, the antibodies can exclude those which are specific to cysteinylated-Cys34 albumin. The ligands can be substantially pure, i.e., purified to at least 90% homogeneity.

Specificity is understood to refer to the relative restriction of antibody or ligand to interactions with a particular antigen(s). While the ligand or antibody could have nonspecific, low affinity interactions with non-antigens, it is understood that the antibody binds selectively, with higher affinity to its target antigen, which in this case is albumin complexed to fatty acids, oxidizing compounds, metal ion, etc., or albumin that has an altered conformation resulting from exposure to ischemic tissues. Further, specificity of antibody to a particular ischemia modified albumin, such as albumin complexed with a certain fatty acid, which may be oxidized, as opposed to other fatty acids, can be useful in identifying the particular ischemic event involved. For example, cardiac ischemia may release one type of fatty acids which complex to the albumin, more than other types of fatty acid.

The invention also comprises a method of determining, in a sample obtained from a subject, an amount of fatty acid that is complexed to albumin, and determining the presence of the ischemic event in the subject based on the amount of fatty acid complexes and a reference value. This method may involve separation of free fatty acid from fatty acid bound to albumin in the subject sample, prior to determination of the amount of fatty acid complexed to albumin. The reference value can be a normal range of fatty acid and albumin complexes present in a non-ischemic control population, and/or a range of fatty acid and albumin complexes in a known ischemic population.

In a variation on this method, the invention comprises the further step of determining the amount of free fatty acid in the subject sample, adding the amount of free fatty acid to the fatty acid complexed to albumin, and comparing the sum to a reference value, to determine whether an ischemic event has occurred. The reference value could be a normal range of the sums of bound and unbound free fatty acid for a population of non-ischemic subjects, or a range of sums of bound and unbound free fatty acid for a population of known ischemic subjects. The free fatty acid determination is preferably performed after separation of free fatty acid from fatty acid bound to albumin.

As used herein, “free” fatty acid refers to fatty acid that is not bound to albumin or fatty acid binding protein or other carrier protein. Bound fatty acid refers to fatty acid bound to albumin, fatty acid binding protein, or other carrier protein. Further, “fatty acid” as used herein refers to any fatty acid, oxidized fatty acid or fatty acid derivative that binds to albumin. As is discussed herein, one to seven fatty acids can bind to albumin, with each complex having a distinct three dimensional configuration that can be specifically recognized and quantitated with a corresponding ligand.

The invention further comprises a method of determining an ischemic event comprising quantitating the relative amount of reduced and oxidized forms of albumin Cys34 in a subject sample. This method may first involve separating the reduced and oxidized forms of albumin Cys34 prior to quantitation. In the alternative, it may involve obtaining two subject samples or dividing a single subject sample into two parts, whereby reduced albumin Cys34 may be quantitated in one sample or part, and oxidized albumin Cys34 may be quantitated in the other sample or part.

The invention also comprises a method having the following steps. First, a determination is made regarding whether a shift in albumin conformation has occurred which would reflect an oxidized Cys34. Next, the results of the first step and a reference value are used to determine whether an ischemic event has occurred. The reference value can be a range of normal values for non-ischemic population, and/or a range of values for a known ischemic population. The shift in conformation can be determined using known methods such as intrinsic fluorescence.

The invention additionally comprises a method of determining an ischemic event by determining the amount of metal ion bound to the albumin metal ion binding sites. This embodiment may include any combination or subcombination of metal ion binding sites, and may specifically include or exclude the N-terminus DAHK binding site and/or the Cys 34 thiol binding site.

While the subject method can use qualitative determination of the existence of two or more ischemia modified albumin markers to determine whether an ischemic event has occurred, it is preferred that at least one of the ischemia modified albumin markers be determined quantitatively. Quantitation can be absolute or relative, i.e., relative to, for example, non-ischemic albumin in the sample.

Advantages of the invention can include a method for evaluating patients with chest pain; ruling out myocardial infarction; diagnosis of brain ischemia or stroke; as an adjunct to other diagnostic cardiac tests for diagnosis of ACS; an immediate method for ruling-out the existence of an ischemic state or event; ruling out asymptomatic ischemia; and a method for detecting placental insufficiency in pregnant women.

As used herein, the terms “ischemic event,” and “ischemic state” mean a local and/or temporary ischemia due to the partial or total occlusion of blood circulating to an organ, or a time point in the disease progression to such an end point.

The following patents and applications relate to ischemia modified albumin and various methods for detecting an ischemic event, and are hereby incorporated by reference in their entireties: U.S. Pat. Nos. 6,461,875; 6,492,179; 5,227,307; 5,290,519; 6,475,743; WO 00/20454; WO 02/096266; US 2004/017554; US 2003/0180820; WO 00/20840; US 2003/0132125; WO 02/089656; WO 04/103150, WO 03/046538, and US-2005-0142613. Further, all references cited herein are incorporated by referenced in their entirety.

### BRIEF DESCRIPTION OF THE DRAWING

FIG. 1 illustrates a mechanism of albumin changes that occur during an ischemic event. A-F designates a number of ischemia modified albumin markers.

### DETAILED DESCRIPTION OF THE INVENTION

A number of terms used herein have the following definitions.

“Albumin N-terminus” refers to the portion of the naturally-occurring albumin comprising of at least the last four amino acids aspartate, alanine, histidine, and lysine. It can include up to 12 N-terminal amino acids.

“Endogenous copper” refers to copper present naturally in a serum or plasma sample. (Note exogenously added during an experiment or test.)

“Excess quantity” of metal refers to the amount of metal that will exceed the amount of binding sites for that metal present in a sample.

“Acute phase reactants” refers to anything the body produces in either an up-regulated or down-regulated fashion in response to a condition, such as an ischemic event or inflammation.

“Sample” means any biological substance containing albumin such as plasma, serum, whole blood, or purified albumin from any source.

“Ischemia” is a form of vascular disease characterized by a decrease in blood supply to a bodily organ, tissue, or part typically caused by constriction or obstruction of one or more blood vessels. Ischemia may also result from vessel rupture, inadequate heart function, low rate of blood flow for a variety of reasons, etc. Inflammation, or numerous other possible symptoms, side-effects, or conditions may also accompany Ischemia. For purposes of this application, any test or procedure that is used to assist in diagnosing, detecting, monitoring, or treating a significant decrease in blood flow to a tissue or organ, or an associated side-effect or symptom of blood flow restriction is considered a method of detecting ischemia. For example, a “test for inflammation” that is performed to assist in diagnosing vessel blockage or obstruction may be considered an “ischemia test”.

While not being bound by any particular theory, it is believed that the present method works by measuring alteration(s) of albumin that occur as a result of oxygen depletion, or ischemia. This process begins when a vessel becomes partially or completely occluded as a result of plaque build up. The build up of plaque may also cause responses consistent with inflammation. These processes begin a cascade of events that release acute phase reactants into circulation. These acute phase reactants are inclusive of, but are not limited to calcium, copper, albumin, fibrinogen, homocysteine, ceruloplasmin, fatty acids (long, medium and short chain), lactate, nitric oxide, molecules containing free radicals and changes in ratios of reduced and oxidized glutathione (Geisel J. et al. (2003) Clinical Chemistry Laboratory Medicine 41(11):1513-7; Morgenstern, I. et al. (2003) Supra; Fellah, H. et al. (2003), supra; Jeremy, J. Y. et al. (2002) Ann. Thorac. Surg. 74(5):1553-7; Lind, P. et al. (2001), Arteriosclerosis, Thrombosis, and Vascular Biology 21:452-467; Kipstein-Grobusch, K. et al. (1999) Br. J. Nutr. 81(2):139-44; and Iskra, M. et al. (1999) J. Trace Elem. Med Biol. 13(1-2):76-81). Albumin is known to scavenge a great number of these acute phase reactants. For example, it is well known that fatty acids bind to albumin (Peters, T, Jr. All about Albumin. Academic Press, Inc. 1996), and that this process is mediated at least in part by calcium concentration (Beck, J. et al. (2004) Anal. Biochem. 325(2):326-336). The binding of three or more long chain fatty acids (LCFA) to albumin changes the conformation of the albumin protein resulting in the opening of sites normally protected from oxidative reactions (Carter, D. et al. (1994) Adv. Protein Chem. 45:153-203). One such site is the cysteine located at position 34. Under normal conditions, i.e., non-ischemic and non-inflammation conditions, Cys34 is located in a hydrophobic pocket that has a depth of 9.5-10 Å (Peters, T, Jr. All about Albumin. Academic Press, Inc. 1996), and is not accessible to molecules greater than the size of a benzene ring. Cys34 is also relatively protected from oxidation by molecular oxygen (Peters, T, Jr. All about Albumin. Academic Press, Inc. 1996). Upon binding three or more LCFA, the pocket opens up and allows other molecules (i.e., those greater than a benzene ring) access to the Cys34 site. Because the Cys34 site is the only free sulfhydryl group in albumin, it is the primary binding site for other endogenous sulfhydryl containing molecules that albumin may be scavenging. These sulfhydryl containing molecules include but are not limited to the reduced and oxidized forms of glutathione, cysteine and homocysteine. It has been shown that there is an inverse relationship between long chain fatty acid binding to albumin and the total albumin sulfhydryl content (Peters, T, Jr. All about Albumin. Academic Press, Inc. 1996). This demonstrates that once LCFA binds Cys34, it is indeed more accessible to oxidative reactions. When Cys34 is in the reduced form (mercaptalbumin) it shows an increased affinity for metal binding at the N-terminus (Zhang Y. et al. (2002) J. Biol. Inorg. Chem. 7(3):327-37; Sengupta S. et al. (2001) Angiology 52(1):69-71). The converse of this phenomenon is true as well; when the Cys34 site of albumin is in the oxidized state (i.e. occupied by another sulfhydryl containing molecule), the affinity for binding metals at the N-terminus is reduced. This binding plays a critical role in determining the extent of metal mediated free radical generation. As an occlusion begins, lack of oxygen flow causes a micro-environment drop in pH. This pH change causes ceruloplasmin to release copper and begin free radical generation cycling. Under healthy conditions, such an effect would be mitigated by albumin scavenging the copper, thereby stopping the cycle. However, if the binding sites for divalent metals on albumin have been altered, the cycle remains unchecked, and Cu is free to generate free radicals which damage other proteins and tissues (Halliwell et al., Free Radicals in Biology and Medicine, 3^{rd} Ed., Oxford University Press, 1999). The dominant products of modified albumin (IMA) produced during a spike of acute phase reactants depends on a number of factors including but not limited to albumin concentration, copper binding on ceruloplasmin at His426, copper released from ceruloplasmin, metal mediated free radical production, fatty acid concentration and homocysteine, cysteine and glutathione concentrations.

Not only does albumin scavenge metals, fatty acids and other acute phase reactants, but albumin is also typically thought of as a sacrifice protein. During oxidative stress it will absorb free radicals in order to protect fragile tissue. Amino acids within the structure of albumin react with free radicals rendering them inactive. Amino acids that are the most susceptible to oxidative damage are methionine, cysteine, tryptophan, tyrosine and histidine. Free radical production is catalyzed by divalent metals such as Cu(II) and H₂O₂ (Sengupta S. et al. (2001) Angiology 52(1):69-71). These types of reactions, called Fenton and Haber-Weiss reactions, are well described in the literature. The generic form of this type of reaction is a reduced metal plus an oxidizing agent reacting to form a reduced metal plus a stronger oxidizing agent. When copper levels are maintained such that there is 1:1 binding of copper to albumin and Cys34 remains in the reduced state (unbound), copper remains redox inactive (Chevion, M. et al. (1993) Proc. Natl. Acad. Sci. 90:1102-1106). Under physiological conditions brought on by ischemia, copper is mobilized resulting in an increase in total copper concentrations in the blood (Berenshtein, E. et al. (1997) J. Mol. Cell. Cardiol. 29(11):3025-34; and Bradshaw R. et al. (1968) J. of Biol. Chem. 243(14):3817-3825). Thus, free radical production by this pathway will only occur under oxidative conditions. Gryzunov (Chevion, M. et al. (1993) Proc. Natl. Acad. Sci. 90:1102-1106) showed that the conformational changes induced by fatty acid binding to albumin facilitated oxidation of Cys34 and initiated redox cycling activity of copper bound to albumin. Nitric oxide (NO) has also been found to play a similar role at the Cys34 binding site of albumin under oxidative stress, thereby indirectly regulating the binding and transport of heavy metals (Sengupta S. et al. (2001) Angiology 52(1):69-71).

Thus, depending on the state of disease progression, it is possible to determine the severity of the ischemic disease by identifying which albumin products predominate following an ischemic event. Early disease could be characterized by copper release, which would be scavenged by albumin and bind predominately at the N-terminus. The details of this mechanism and techniques for measuring this product are provided in patent application US2003/0180820. Fatty acid concentrations would increase during early stages of the disease pathway. The fatty acids would then bind to albumin, inducing conformational changes and exposing the Cys34 site, which would cause it to become more susceptible to oxidative reactions. The conformational change induced by fatty acid binding may be measured and quantified as one indicator of disease progression. The next phase in the disease process would involve oxidative reactions taking place at the now exposed Cys34 site on albumin. Relevant oxidative reactions can be measured either directly or indirectly by quantifying the reaction products. For example, cysteine, homocysteine or glutathione bound at the Cys34 site would be indicative of an oxidative reaction. Upon occupying the Cys34 binding site, albumin undergoes a conformational change resulting in a decreased metal binding affinity at the N-terminus and perhaps other sites.

Decreased binding at the N-terminus can be due to conformational changes of the terminal DAHK residues, whereby divalent metal binding is decreased. Decreased binding at the N-terminus can also be due to formation of 2-oxo-histidine at residues 3 and/or 9 of the N-terminus. Histidine has been identified as an amino acid residue that participates in the binding of transition metals such as copper and cobalt. During oxidative stress, as induced by many conditions such as ischemia or inflammation, histidine will undergo oxidative reactions forming 2-oxo-histidine. The susceptibility of histidine is dependent on the sequence, structure and geometry of albumin.

In the presence of ascorbic acid, these oxidative reactions are catalyzed by transition metals that are present in the blood, loosely bound to albumin or other metal carrying proteins (Schoneich et. Al. (2000) Journal of Pharmaceutical and Biochemical Analysis 21: 1093-1097). Hydroxy free radicals form adducts (Yim et. al. Pro.c Natl. Acad. Sci. 87: 50006-5010) with the aromatic ring being the most susceptible (Stadtman et al. (1993) Ann. Rev. Biochem. 62: 797-821). 2-oxo-histidine is relatively unstable (Lewisch et al (1995) Anal. Biochem. 231:440-446) and under some conditions will continue reacting to form aspartic acid and/or arginine rendering that site incapable of binding transition metals with high affinity.

As discussed in WO 00/20840, reduced binding at the N-terminus can also be due to N-terminal truncation and acetylation.

Other sites on albumin may also be affected by ischemia. In addition to the N-terminus, there are several other well-established metal binding sites and exposed amino acid residues that are susceptible to oxidative damage. These sites include His9, His18 (Bradshaw, R. et al. (1969) J. Biol. Chem. 244(20):5582-5589; and Shaw C. F. et al. (1984) [CITATION PLEASE]) and sites that include the participation of thiols contributed by Cys34 (Bal et al. (1998) J. Inorg. Biochem. 70:33-39). Bal et. al. (Demant, E. et al. (2002) Biochem. Journal. 363(Pt 3):809-815; and Takabayashi, K. et al. (1983) Eur..l Biochem. 136:291-295), speaks to binding sites in domain I, specifically around His105 and His146, and domain II, specifically around His247. These sites may be easily modified under oxidative conditions when the conformation of albumin is such that these sites are exposed. All histidine sites on albumin can be oxidized to 2-oxo-histidine, which may in turn continue to react to form aspartic acid and/or arginine.

Utilizing multiple detection schemes and incorporating different detection methods will enable each marker to be identified quantitatively such that the individual marker or a combination of the individual markers can be used to diagnose not only the ischemic disease, but the state of progression of the disease as well.

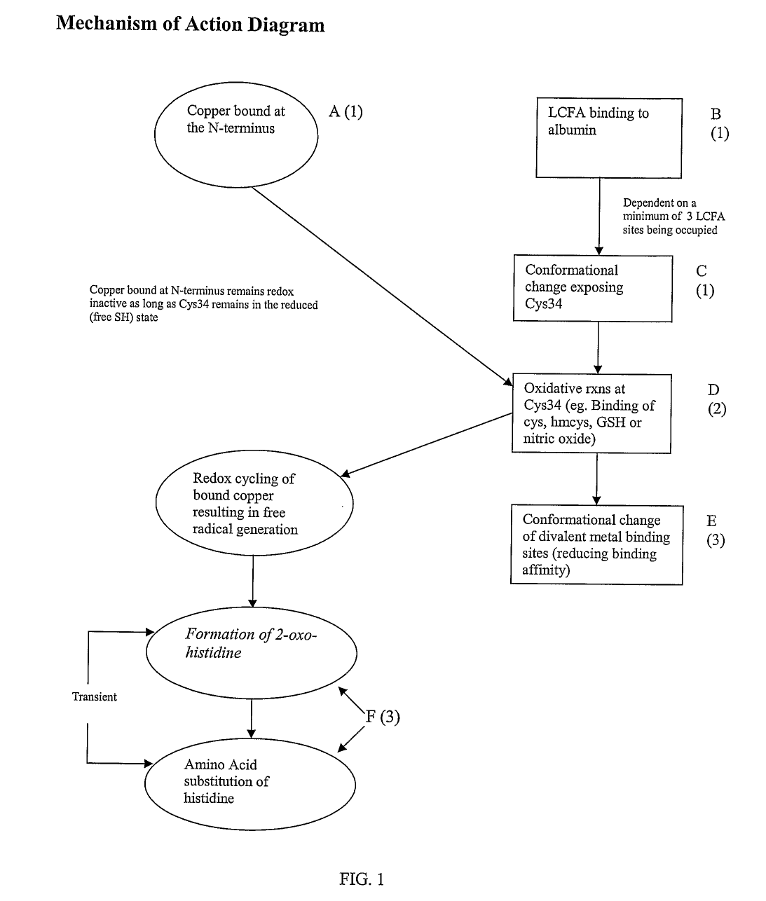

The ischemia markers can be combined in various algorithms to yield a product value that is useful in the diagnosis of the ischemic event and/or the determination of the progress of the ischemic event. In such algorithm, each of the markers can be assigned a “severity factor” which is a value that reflects the weight that a particular marker has as an indicator of an ischemic event. Additionally, the algorithm may take into account the concentration of the marker in the patient sample as an “intensity factor”. FIG. 1 sets forth some of the various ischemic markers described above. Marker A is Cu bound at the N-terminus of albumin. Marker B is long chain fatty acid (one to five) bound to albumin. Marker C is albumin that has undergone the conformational change resulting from fatty acid binding. Cys34 becomes exposed upon the binding of at least three fatty acids. Marker D is any one of a number of albumins that have been oxidatively modified at Cys34, including albumin modified at residue 34 with Cys, Homocysteine, GSH or nitric oxide. Marker E is albumin that has reduced divalent metal binding at the N-terminus or elsewhere due to conformational changes caused by modification by binding at Cys34. Marker F is 2-oxo-histidine or amino acid substitution of histidine.

In one embodiment (FIG. 1), markers A-F have been assigned different severity factors, with A-C having severity factors of 1, D having a severity factor of 2 and E and F having severity factors of 3. Again, assignment of such severity factors reflects the practitioner's understanding of the relative significance of the marker as an indicator of the severity of the ischemic event. As understanding of ischemic events and particular markers improve, different severity factors can be assigned to existing and new markers.

A detailed description of one embodiment of an algorithm using the markers set forth in FIG. 1 is set forth in Examples 7 and 8.

It should also be understood that the combination of the results of the subject method and the patient's report of duration of symptoms, can be useful in informing the physician of the most appropriate therapeutic treatments. It is common for an ischemic event to develop over a number of hours, during which events at the cellular level shift from ischemia with relatively no cell death to severe ischemia and massive necrosis. If events evolve over the period of several hours, they can be tracked using repeated applications of the present method, and appropriate treatments can be prescribed to reduce or avert necrosis. Additionally, if the ischemic event evolves to severe ischemia very rapidly, i.e., over a matter of minutes, the present method can be useful in quickly diagnosing the severity of the situation, and when used in combination with the patient's report of symptom duration, the subject method can again inform the physician of most appropriate treatment options.

Examples of methods for detecting and quantitating ischemic markers, and for combining such data in an algorithm are given below.

### EXAMPLE 1

### Immunoassay Detection of IMA

This invention can utilize an immunoassay method of detection in which antibodies to each form of modified albumin can be made and used either as a cocktail or as individual antibodies. In this case an antibody would be generated to (a) the complex of Cu bound to the N-terminus of albumin; (b) the structure of one or more fatty acids bound to albumin; (c) the induced conformational change resulting in the exposure of the Cys34 site on albumin; (d) products of oxidative reactions at the Cys34 site, which include but are not limited to glutathione and homocysteine bound at Cys34; (e) the conformation change of albumin induced by sulfide containing molecules at the Cys34 site resulting in decreased binding of divalent metals at sites including but not limited to the N-terminus; (f) the altered divalent metal ion binding sites; (g) additional oxidative reactions taking place at the N-terminus as a result of free-radical production, which include but is not limited to 2-oxo-histidine where the histidine is located at position 3 and/or 9 of albumin, and amino acid substitution of histidine.

Generation of antibodies can be carried out using the IMA targets, as specified above, as antigens. Various techniques, such as synthetic peptide phage display and antibody/peptide phages, using either monoclonal or polyclonal antibodies, antibody Fab and Fc fragments, can be used to obtain the desired ligands. Antibodies can be raised from animals or by other methods such as phage display. Antibodies or ligands used in the measuring step are labeled, preferably with an enzyme, fluorescent label or other detectable tags commonly used in immunoassay.

More specifically, the generation of antibodies can be directed to albumin metal complexes. Albumin is known to have multiple sites that bind metal ions such as copper, nickel, zinc, cobalt and others. One such binding site is the N-terminus of albumin, but other binding sites have also been established (Bal et al. (1998) J. Inorg. Biochem. 70:33-39). These sites include the N-terminus His3, His9, His18, Cys34, His105, His146 in domain I, and His247 in domain II. These sites are susceptible to oxidative damage induced by an ischemic event. Antibodies to these metal-occupied binding sites and/or oxidatively damaged epitopes (amino acid sequence and the related dimensional structure) in the native state, and the epitope changes caused by exogenously adding metal ions, or similar epitope motifs and/or synthetic peptides can be produced.

Albumin in a sample scavenges endogenous copper. During oxidative stress, copper is utilized in Fenton-type reactions resulting in site-directed oxidative damage to albumin (Christodoulou, J. et al. (1994) Eur. J. Biochem. 225(1): 363-368). This same type of reaction can be created in Vitro incubating native albumin and exogenous copper and ascorbic acid. Antibodies to albumin and/or oxidized albumin domains, and/or motifs, and/or synthetic peptides can be produced.

Fatty acids (long, medium and short chain) bind to albumin thereby changing its conformation. Although albumin contains 6-7 long chain fatty acids (LCFA) binding sites, upon binding 3-5 LCFA, the metal binding capacity of albumin decreases (Kashiba-Iwatsuki, M. et. al. (1997) Arch. Biochem Biophys. 345(2):237-42). This is partially because fatty acid binding enhances the ascorbic acid oxidizing activity of albumin-bound copper (Christodoulou, J. et al. (1994) Eur. J. Biochem. 225(1): 363-368). Antibodies can be produced to detect fatty acid bound to albumin and fatty acid bound albumin domains, motifs and peptides.

Fatty acid binding to albumin results in the opening of sites normally protected from oxidative reactions. Cys34 is one such site. Once Cys34 is exposed, it becomes more accessible to react with other sulfhydryl containing molecules, such as homocysteine, cysteine and glutathione. It has been shown that when Cys34 is in a reduced form, the metal binding capacity at the N-terminus is increased. Inversely, when Cys34 is coupled with another sulfhydryl-containing molecule, the N-terminus metal affinity is reduced. The antigen strategy in this case would be to identify (by, e.g., X-ray crystallography or NMR) the albumin Cys34 and N-terminus region sequence and the tertiary structural shifts caused by fatty acid bound to albumin, and design a corresponding motif or peptide to mimic the shifts.

Cys34 is the only free sulfhydryl group in albumin. Nitric oxide binding at this site has been found to play a role in regulating the binding and transport of heavy metals (Sengupta S. et al. (2001) Angiology 52(1):69-71). Alkylation, glycation, oxidation or nitrosylation of albumin at the Cys34 site will result in a change in metal binding (Christodoulou, J. et al. (1994) Eur. J. Biochem. 225(1): 363-368). Modifications of albumin, albumin domains and or the Cys34 region motif and synthetic peptides can be used as antigenc targets.

As a preferred method of detecting the IMA discussed above, immunoassay methods would use ligand(s) specific to the antigens comprising the above conformers. To produce peptide phage display ligands, DNA phage display libraries, which represent the conformers of interest, are constructed and screened against the albumin modifications using ELISA techniques. Following multiple rounds of selection, peptides corresponding to the positive phages are synthesized and immobilized onto a solid support, where IMA in the sample will migrate and attach to the ligands. A secondary antibody with an attached label, such as horseradish peroxidase or another commonly used reporter enzyme that reacts with the substrate, is added to yield detectable products. Another approach would be screening the modifications to IMA conformers' phage libraries against the antibody or antibody fragment phage libraries. This would allow the selection of the ligand that matches the IMA conformers.

Another approach for detecting modifications to albumin is to produce monoclonal and polyclonal antibodies or antibody mixtures for use in the subject methods. The generation of antibodies by these methods is understood by those in the field.

The sample screening methods for determining whether or not and to what extent ischemia is present would include, but is not limited to (1) sandwich ELISA assays to capture the described modifications and (2) other competitive immunoassay to completely capture the naturally-occurring albumin and the disease induced modifications.

The immunoassay detection schemes of this invention may be applied to any platform that is commercially available or that is specifically designed for said diagnostic test.

### EXAMPLE 2

### Detection of FFA Binding to Albumin by Spectroscopic Methods

Plasma or serum samples from a positive ischemia population have more fatty acid (FA) bound per albumin molecule, relative to that of samples from an apparently healthy, normal population. This observable event will be exploited to use as one arm in a detection scheme for the diagnosis of ischemia. FA binding to albumin can be measured qualitatively and/or quantitatively. One method is described below.

First, sample is moved through a column or over a plate or well, containing or lined with fluorescent-labeled fatty acid binding proteins (FABP). FABP may be purchased commercially or made specifically for this application. Free fatty acid (FFA) in the sample binds to the FABP and is quantified by fluorescence measurements using a modern/standard fluorometer, as understood by those familiar with the art.

The first step in the process, as described above, will yield (as eluent or supernatent) a FFA-free sample. FA bound to protein remains in the sample, and is primarily bound to albumin. FA is dissociated from albumin under conditions and by methods described by those familiar with the art (Demant, E. et al. (2002) Biochem. Journal. 363(Pt 3):809-815; and Takabayashi, K. et al. (1983) Eur..l Biochem. 136:291-295). The dissociated/unbound FA is measured using a FABP. This may or may not be the same FABP that was used in the first step. The process may be carried out by (1) adding the FABP to the sample solution, by (2) running the sample over a column or plate modified with the FABP, or by (3) other methods that combine the FABP and the sample. Free FA binds to the FABP and is quantified by fluorescence measurements using a modern/standard fluorometer.

Analysis of the FA binding to albumin, as it relates to the degree of the disease state, may be done by one of more of the following methods. (1) Only the amount of FFA bound to albumin (as measured in step 2 by first removing it from the protein) is measured. The amount of FA bound to albumin is compared to a predetermined calibration and/or an ischemia evaluation scale. (2) The sum of the FFA measured in steps 1 (FFA) and 2 (FA bound to albumin) is compared to a pre-determined calibration and/or an ischemia evaluation scale. (3) It may or may not be necessary to normalize either (1) or (2), or both (1) and (2), for the concentration of albumin in the sample (as measured by standard techniques understood by those in the art).

The use of free fatty acids as an indicator of an ischemic event is known (U.S. Pat. No. 5,227,307). Such methods, however, do not identify the complex of fatty acid and albumin as a marker for ischemia.

### EXAMPLE 3

### Detection of IMA, Conformational Change of Albumin Following FFA Binding to Albumin by Spectroscopic and Isoelectric Focusing Methods

Samples from a positive ischemia population have more FA bound per albumin molecule, relative to that of samples from an apparently healthy, normal population. Therefore, samples from the positive ischemia population will also show increased albumin conformation changes resulting from the FA being bound to albumin. This is another arm of a detection scheme to aid in the diagnosis of ischemia. Conformational changes to the albumin molecule, which result from FA binding, can be measured (qualitatively and/or quantitatively) by several methods. Two different methods are described below.

1. Determination by Isoelectric Focusing Methods
Conformational changes to albumin, resulting from FFA binding to the protein, can be determined using isoelectric focusing methods (based on work reported from Gryzunov, Y. et al. (2003) Arch. Biochem. Biophys. 413(1):53-66. Isoelectric profiles of HSA (and modifications there-of) demonstrate shifts toward the more acidic albumin isoform (i.e. a shift to a lower pI) as the molar ratio of fatty acid:HSA increases. Samples from a positive ischemic population can be distinguished from a nonischemic population, based the relative amount of protein focused at a “standard” pI to that at the “shifted” pI.
Electrophoresis of a sample is conducted according to standard isoelectric focusing techniques understood by those in the field. In general, the “sample” is subjected to a strong electric field in a medium with a pH gradient specifically designed for the protein(s) under investigation. Appropriate experimental conditions for the detection scheme are consistent for testing. During the testing time, each protein migrates to and concentrates in a position where the pH is the same as its isoelectric pH. Proteins are precipitated in place on the gel with an appropriate precipitating agent, stained with an appropriate dye to make their position more visible, and the dye peaks are scanned using spectrophotometric methods. For example, a densitometer is used to measure and plot the absorbance as a function of position along the gel. The relative absorbance ratios of the proteins in the “standard” and “shifted” pI positions are used to determine whether a sample is normal or elevated above a predetermined cut-off or along an ischemia evaluation scale.

2. Determination by Changes in Intrinsic Fluorescence
Conformational changes to albumin, resulting from FFA binding to the protein, can be determined by looking at the changes in the intrinsic fluorescence of the protein (based on work done by Takabayashi, K. et al. (1983) Eur..l Biochem. 136:291-295 and Aguanno, J. et al. (1982) J. Biol. Chem. 257(15):8745-8782. When FAs are bound to albumin, the wavelength for the maximum fluorescence emission of the complex is shortened, in a concentration dependent manner. Normal samples therefore, have less wavelength shift than samples from a positive ischemia population.
Another example of a detection scheme takes advantage of the proximity of Tyr30 to Cys34 on the albumin molecule. Conformational changes in albumin, resulting from FFA binding to the protein causes the Cys34 pocket to open/loosen, exposing Cys34 to subsequent reactions. Accompanying this change is the exposure of Tyr30, which yields an increase in fluorescence intensity, compared to a sample with little or no conformational changes resulting from FFA binding to albumin. For this scheme, fluorescence is measured by methods used by those skilled in the art.

### EXAMPLE 4

### Detection of IMA, Increased Oxidation of Cys34

During a state of ischemia, increased binding of FFA to albumin leads to changes in conformation of the protein. These conformation changes, in turn, result in increased accessibility to Cys34 for oxidizing agents. Thus, samples from a positive ischemia population have a higher percentage of oxidized Cys34, relative to that of samples from an apparently healthy, normal population. The oxidized Cys34 may be expressed as several different forms. Measuring the degree of oxidation activity at Cys34 is another arm of a detection scheme to aid in the diagnosis of ischemia.

Oxidation of Cys34 that results from an ischemic event can be detected by several methods, both indirectly and directly. For example, the amounts of reduced and oxidized forms of albumin Cys34 can be determined using electrospray ionization mass spectrometry (Fabisiak, J. et al. (2002) Antioxidants and Redox Signaling. 4(5):855-865); oxidative modifications to albumin Cys34 can also be quantified using a fluorescence-based SDS-Page Assay (Kadota, K. et al. (1991) Japanese Circulation Journal 55:937-941) (Fabisiak, J. et al. (2002) Antioxidants and Redox Signaling. 4(5):855-865, described such a method using the fluorogenic agent, ThioGlo-1); or the redox activity at Cys34 can be monitored using a modified electrode containing a redox mediator. The details of another example are described below.

The relative amounts of reduced and oxidized forms of albumin Cys34 contained in a sample can be determined by separating the two forms using HPLC, followed by subsequent spectrophotometric (or other) detection (Kagan, V. et al. (2001) Hypertension in Pregnancy 20(3):221-241). Conditions of the HPLC separation are specific for the needs/requirements of this application and are consistent with techniques used by those skilled in the art. The peaks corresponding to the two different redox forms of albumin Cys34 have been identified and elute at different times. The eluent is monitored and the relative amounts of oxidized and reduced Cys34 albumin measured. The measurement may be done by a number of techniques, including, but not limited to spectrophotometrically, electrochemically, or other. If needed, the total reduced thiol content of the sample can be measured by a version of the Ellman method or other standard measures of thiol content, as understood by those familiar with the art.

### EXAMPLE 5

### Detection of IMA, Albumin Conformational Changes Resulting from Cys34 Oxidation or Other Cys34 Binding Modifications

Oxidation or other binding modifications of albumin Cys34 results in conformational changes to the protein, which can be determined by looking at the changes in the intrinsic fluorescence of the protein (Aguanno, J. et al. (1982) J. Biol. Chem. 257(15):8745-8782; and Beck, J. et al. (2004) Anal. Biochem. 325(2):326-336). For example, when Cys34 is oxidized and a conformation change occurs, the wavelength for the maximum fluorescence emission is shortened, in a concentration dependent manner. Normal samples therefore, have less wavelength shift than samples from a positive ischemia population.

### EXAMPLE 6

### Detection of IMA, Decreased Metal Binding

A state of ischemia leads to changes in the metal binding capacity of albumin. This event can be quantified or qualified and used as another arm of a detection scheme for ischemia. Samples from a positive ischemia population would have proportionally less metal bound to albumin than that of a sample from an apparently healthy, normal population.

The level of metal binding to albumin can be detected, directly and/or indirectly, by a number of methods, including, but not limited to the following: (1) isothermal titration calorimetry (Zhang Y. et al. (2002) J. Biol. Inorg Chem. 7(3):327-37), (2) equilibrium dialysis, or (3) utilizing metal chelators (which may be commercially available or designed specifically for these purposes). As another example, extrinsic metal ion is added to a sample, allowed time to bind to albumin, and the unbound remaining metal is quantified to determine the metal binding capacity of the sample. The quantification may be based on a number of analytical methods, including, but not limited to, spectrophotometrically (either directly or indirectly), electrochemically, fluorometrically if the metal is labeled as such. Several of these methods are described in patents (U.S. Pat. No. 5,227,307; U.S. Pat. No. 6,461,875; and U.S. Pat. No. 6,492,179) and patent applications (WO 00/20840) assigned to the assignee of the subject application. As a third example, the metal binding capacity may be measured using a designer metal that has detectable properties that change upon binding to albumin.

Any of the detection schemes or combination of detection schemes may be used to determine whether or not the patient is ischemic, and may also be used to determine the state or severity of the disease progression.

### EXAMPLE 7

### Algorithm for Determination of Severity of Ischemic Event

A number of algorithms for calculation of a score that is indicative of the occurrence or non-occurrence of an ischemic event are possible. The exact form of the algorithm can change to give greater or lesser weight (“severity factor”) to ischemic markers A-F of FIG. 1 or other ischemic markers, as more information is obtained about the sequence, occurrence and importance of such markers during an ischemic event. The algorithm can also include a concentration parameter or “intensity factor” in the calculation of the ischemia score. Normal (i.e., non-ischemic) populations as well as ischemic patients are studied to determine normal range and mild, moderate and severe ischemia ranges.

In one embodiment, the algorithm takes the form of the following equation:

(SF)^{A}(IF)^{A}+(SF)^{B}(IF)^{B}+(SF)^{C}(IF)^{C}+(SF)^{D}(IF)^{D}+(SF)^{E}(IF)^{E}+(SF)^{F}(IF)^{F}=IS

where SF is severity factor, IF is intensity factor, superscripts A-F represent markers A-F, and IS is the ischemia score. Markers A, B and C are assigned SFs of 1 and marker D is assigned a SF of 2 based on current understanding of the importance of these markers as indicators of an ischemic event. The IF could be an actual concentration or could be a digit that reflects a predetermined concentration range. For example, an IF of 1 could reflect 1-5 ng/ml in serum, and an IF of 2 could reflect 5-8 ng/ml in serum.

This algorithm allows for multiple combinations of modifications to be factored into the ischemia score, talking into account the severity factor for the marker and its concentration.

In this embodiment, the ranges for mild, moderate and severe ischemia are as follows:

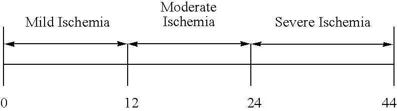

Thus, if a patient had positive identification of modifications A, B, C and D and the intensity of each modification is measured as 4, 4, 3, 1, respectively, then the overall ischemia score would be calculated as:

(1)(4)+(1)(4)+(1)(3)+(2)(1)=13

Because the modifications are in early stages in the pathway, it is indicative that the ischemia is less severe, but the intensities (concentrations) of the early modifications are higher. The overall ischemia score shows that this patient is oil the borderline of mild and moderate ischemia.

### EXAMPLE 8

### Algorithm for Determination of Ischemic Event

If a patient is found to have a positive identification of modifications A, B, C, D, E and F (E and F having a SF=3), and the intensity of each modification is measured as 1, 1, 2, 3, 4, 4, respectively, then using the algorithm of Example 7, the overall ischemia score would be:

(1)(1)+(1)(1)+(1)(2)+(2)(3)+(3)(4)+(3)(4)=34

In this case, the modifications span the entire spectrum of the pathway, but the higher intensities lie with the modifications farther into the pathway, indicating that the ischemia is more advanced. The overall ischemia score, in this case, indicates that this patient has moved well into the severe ischemia area.

1. A method comprising:
(a) determining, in a sample material obtained from a subject, at least two ischemia modified albumin markers selected from the group consisting of complexes of fatty acids bound to albumin (marker B), albumin molecules with open Cys34 sites (marker C), albumin molecules that are products of oxidation at Cys34 (marker D), albumin molecules with altered conformation or altered or reduced divalent metal binding due the conformational change or oxidation at Cys34 (marker E), and albumin molecules that have been oxidized at the N-terminus (markers A and F);
(b) determining at least one ischemia score based at least in part on results of the determining of the at least two ischemia modified albumin markers of step (a); and
(c) determining the presence of an ischemic event in the subject based at least in part n the at least one ischemia score.

2. The method of claim 1, wherein step (a) further comprises:
(a)(1) measuring the concentration of the ischemia markers and providing an intensity factor that corresponds to that concentration;
and wherein step (b) further comprises:
(b)(1) providing a severity factor for each ischemia marker;
(b)(2) combining the severity factor and the intensity factor for each marker in an algorithm, whereby the ischemia score is produced.

3. The method of claim 2, wherein said algorithm of step (b)(2) comprises:
(SF)^{A}(IF)^{A}+(SF)^{B}(IF)^{B}+(SF)^{C}(IF)^{C}+(SF)_{D}(IF)^{D}+(SF)^{E}(IF)^{E}+(SF)^{F}(IF)^{F}=IS
where SF is severity factor, IF is intensity factor, superscripts A-F represent markers A-F, and IS is the ischemia score.

4. The method of claim 1, wherein the products of the oxidative reaction at Cys34 of albumin are selected from the group consisting of Cys34 bound to glutathione, homocysteine, cysteine and nitric oxide.

5. The method of claim 1, wherein the albumin molecules with reduced metal binding due to the conformational change at Cys34 have reduced binding at the N-terminus.

6. The method of claim 1, wherein the albumin molecule which has been oxidized at the N-terminus is an albumin that is 2-oxo-histidine at His3 or His9.

7. The method of claim 6, wherein the 2-oxo-histidine converts to aspartic acid or asparagine.

8. The method of claim 1, wherein the albumin molecule that has been oxidized at the N-terminus is an albumin that is bound to Cu ion at the N-terminus.

9. A method comprising:
receiving at least one ischemia score, and
determining the presence of an ischemic event in a subject based at least in part on the at least one ischemia score;
wherein the ischemia score has been determined based at least in part on results of a determination, in a sample obtained from the subject, of at least two ischemia modified albumin markers selected from the group consisting of complexes of fatty acids bound to albumin(marker B), albumin molecules with open Cys34 sites (marker C), albumin molecules that are products of oxidation at Cys34 (marker D), albumin molecules with altered conformation or altered or reduced divalent metal binding due to the conformational change or oxidation at Cys34(marker E), and albumin molecules that have been oxidized at the N-terminus (markers A and F).

10. A ligand that is specific to a complex of fatty acid and albumin.

11. A ligand that is specific to an albumin molecule with an exposed Cys34 site.

12. A ligand that is specific to an albumin molecule that is a product of an oxidation reaction at Cys34.

13. A ligand that is specific to an albumin molecule that has an altered conformation or reduced divalent metal binding due the conformational change at Cys34.

14. A ligand that is specific to an albumin molecule that has 2-oxo-histidine at His3 and/or His9.

15. A method comprising:
(a) determining, in a sample obtained from a subject, an amount of fatty acid that is complexed to albumin in the sample;
(b) determining the presence of an ischemic event in the subject based on the determination of step (a) and a reference value.

16. The method of claim 15, further comprising the step of:
(c) determining the amount of free fatty acid in said patient sample, adding it to the fatty aid complexed to albumin, and comparing the sum to a reference value, to determine whether an ischemic event has occurred.

17. The method of claim 15, wherein the free fatty acid, or fatty acid complexed to albumin which is dissociated from the albumin, is determined by conducting spectroscopic methods on the fatty acids.

18. The method of claim 17, wherein the spectroscopic method is fluorometry.

19. The method of claim 15 wherein the amount of fatty acids complexed to albumin is determined by a shift in isoelectric focusing (pI) of the complex as compared to a pI for the albumin.

20. The method of claim 15, wherein the amount of fatty acids complexed to albumin is determined by the intrinsic fluorescence of a Tyr30 which is revealed upon fatty acid binding to albumin.

21. A method comprising:
(a) quantitating the relative amounts of reduced and oxidized forms of albumin Cys34; and
(b) determining an ischemic event in the subject based on the quantitation of step (a) and a reference value.

22. The method of claim 21, wherein the reduced and oxidized forms of albumin Cys34 are first separated prior to step (a), and such separation is accomplished by high performance liquid chromatography.

23. The method of claim 21, wherein step (a) is accomplished by a method selected from the group consisting of spectrophotometric, electrochemical and chemical methods.

24. A method comprising:
(a) determining whether a shift in albumin conformation has occurred which would reflect an oxidized Cys34; and
(b) determining from results of step (a) and a reference value whether an ischemic event has occurred.

25. The method of claim 24, wherein the shift in conformation is measured by intrinsic fluorescence of the albumin.

26. A method of detecting or measuring an ischemic event comprising the steps of:
determining the amount of metal ion bound to the albumin metal ion binding sites.

27. The method of claim 26, wherein the determining step is selected from the group consisting of isothermal titration calorimetry, equilibrium dialysis, and use of chelating agents.

28. The method of claim 26, wherein the determining step comprises:
(a) adding excess metal ion to the sample;
(b) allowing the metal ion to bind to the albumin binding sites; and
(c) quantitating the unbound metal ion to determine the metal binding capacity of the albumin, wherein said quantitation is accomplished spectrophotometrically, electrochemically, fluorometrically.

29. The method of claim 26, wherein the amount of metal ion bound to the binding sites is an absolute determination.
